(19) United States
(12) Patent Application Publication
(10) Pub. No.: US 2016/0095162 A1
(43) Pub. Date: Mar. 31, 2016

(54) UNIVERSAL SYSTEM FOR MEASUREMENT INSTRUMENTS FOR INDUSTRIAL HYGIENE AND ENVIRONMENT

(71) Applicant: Alexandre Fascina Da Silva, Sao Paulo (BR)

(72) Inventor: Alexandre Fascina Da Silva, Sao Paulo (BR)

(21) Appl. No.: 14/864,175

(22) Filed: Sep. 24, 2015

Related U.S. Application Data
(60) Provisional application No. 62/055,296, filed on Sep. 25, 2014.

(51) Int. Cl. H04W 84/18; G01N 33/00

(52) U.S. Cl. CPC H04W 84/18; G01N 33/0004

(57) ABSTRACT

UNIVERSAL SYSTEM FOR MEASUREMENT INSTRUMENTS FOR INDUSTRIAL HYGIENE AND ENVIRONMENT, consisting of a Data Collector (1), this equipment will be a portable device or computer equipped with Microsoft Windows or Android Operating System, used to communicate with following instruments: Wet bulb globe temperature Thermometer Index (WBGT) (2), Sound level meter (3), Noisedosimeter (4), Vibration meter for Hand, Arm and Whole Body (5), Fixed type portable gas detectors (6), Alpha, beta, gamma and/or Neutrons ionizing radiation detector (7), Non-ionizing radiation detector(8), Lux meters (9), IAQ Systems—Internal air quality (10) and IAQ Systems—Internal water quality (11). Its application field belongs to the area of Occupational Hygiene applicable to industries or businesses in general in which workers are exposed to physical, chemical and biological agents causing occupational diseases.

### CROSS-REFERENCE TO RELATED APPLICATIONS

This application claims the benefit of priority to U.S. Provisional Application No. 62055,296 filed Sep. 25, 2014, the contents of which are incorporated herein by reference.

### FIELD OF THE INVENTION

The present invention belongs to the area of Occupational Hygiene applicable to industries or businesses in general in which workers are exposed to physical, chemical and biological agents causing occupational diseases. Meeting the legal requirements by the Regulatory Standards, ACGIH—American Conference of Industrial Hygienists and others Standards of Occupational Hygiene. As well as international requirements, according to OSHA, NIOSH valid in the USA and Canada and the European directives.

### BACKGROUND OF THE INVENTION

Portable gas monitor capable of measuring from 01 up to 06 gas types simultaneously coupled with an accelerometer and a wireless radio.

The innovation of our gas monitor against current instruments is that they do not have the coupling of the functions of an accelerometer and a wireless radio which adds improvement to the function of the watchman of the confined space because it will allow him to monitor the worker outside the confined space through a device developed by CHROMPACK or through a Windows or Android computer. These couplings will allow the watchman to confirm if the worker is moving, his position: standing, sitting or lying down whenever he is within any confined space where visibility is impaired by geography and geometry of the site.

These features are intended not only to measure the concentrations of gases, but also to provide reliable tools to the watchmen and first responders to identify risk situations such as sudden illness of workers and current concentrations present in the confined space in which workers are exposed to even before entering the confined space.

The new real gain of this technology is precisely to allow whoever is monitoring confined space, to make the right decision and use the required Personal Protection Equipment—PPE to perform the rescue or relief to workers when necessary.

There are no instruments designated to Occupational Hygiene (or Industrial) and Environment in the global market with wireless technology measurement with the various functions intended for Occupational and Environmental Hygiene as broad and straightforward as the patent now in matter is committed to perform.

### SUMMARY OF THE INVENTION

Observing the drawbacks mentioned above and the increase of safety of users, after several studies and research, the UNIVERSAL SYSTEM FOR MEASUREMENT INSTRUMENTS FOR INDUSTRIAL HYGIENE AND ENVIRONMENT, the object of this patent was developed in an intelligent and unusual way. Along with precision instruments it will be able to provide clear benefits to workers exposed to physical, chemical and biological hazards in their daily routine.

Thus bringing a number of advantages and benefits, amongst them:

Information of real-time measurements;

Centralized and integrated information;

No need of great infrastructure for equipment installation;

More comfort for workers using the equipment due to the small size of instruments;

Highly precise compatible instruments and fair prices

Designed to meet the requirements for International-standards,

Objective possibility to export high technology due to the cost-benefit of these excellent, versatile instruments.

In order to have a better view of the unprecedented UNIVERSAL SYSTEM FOR MEASUREMENT INSTRUMENTS FOR INDUSTRY AND ENVIRONMENT HYGIENE, attached is a drawing reference with more detailed information:

### BRIEF DESCRIPTION OF THE DRAWINGS

FIG. 1 Illustrates a communication between the data collector and measurement instruments through wireless technology according to an embodiment of the present invention.

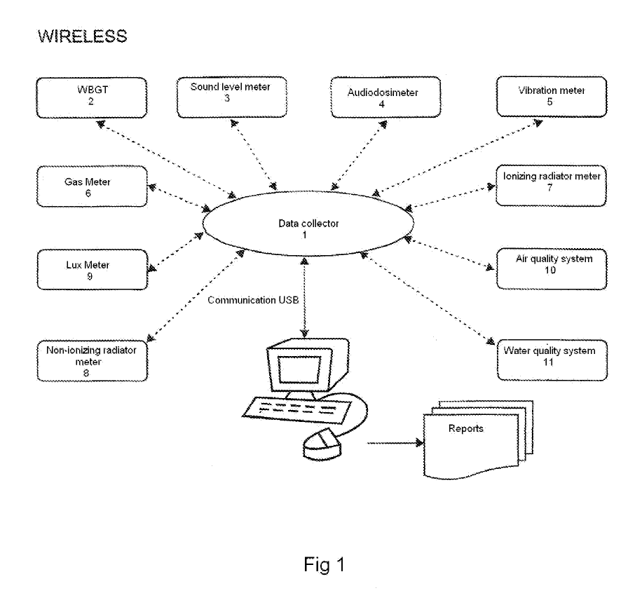

### DETAILED DESCRIPTION OF THE INVENTION

Universal system for measurement instruments for industrial hygiene and environment comprises: a Data Collector (1), this equipment is a portable device or computer equipped with Microsoft Windows or Android operating system used to communicate with following instruments: a Wet bulb globe temperature Thermometer Index (WBGT) (2), a Sound level meter (3), an Audio dosimeter (4), a Vibration meter for Hand, an Arm and Whole Body (5), a Fixed type and portable gas detector (6), Alpha, beta, gamma and/or neutrons ionizing radiation detector (7), a Non-ionizing radiation detector (8), a Lux meters (9), Systems IAQ—Indoor air quality (10), and Systems WQ—water quality (11).

The communication between the controller and measurement instruments is performed through wireless technology. With this equipment it is possible to simultaneously monitor in real time the information of heat, noise, gas concentration, vibration, non-ionizing radiation, ionizing radiation, illumination, etc. in which workers are exposed to.

The data collector runs on a software developed by Chrompack which makes communication possible.

Wet bulb globe temperature Thermometer Index (WBGT): The index of measurement of the wet bulb globe thermometer—WBGT (included in ISO7943 and ISO7726). The measurement system consists of three thermometers called dry bulb, wet bulb and globe, they make up this index and may be added to humidity sensors, wind speed and storage in electronic memory of records of worker's entries and exits from the working area where the instrument is located, thus showing the actual exposure to the heat. This instrument has the following features:
a) It records at every 1 minute average values of dry bulb temperature, wet bulb temperature, globe temperature, WBGT internal calculation, WBGT external calculation, humidity, wind speed and time; date and time.
b) Registration formats: Date (DD/MM/YYYY), and Time (HH:MM:SS) according to entry and exit of worker in the area monitored by the instrument. Registration is done through electronic badge that identifies each worker individually.
c) Wireless communication with the data controller in order to monitor real time information.
d) Operates using 9V alkaline battery.

3) Sound level meter: Also known as Sound Pressure level Meter, or “decibel” widely used by state environmental agencies alike as well as other City departments. This same instrument is used in environmental and working safety training courses at schools and universities.

Besides normal functionality this instrument will add to this type of instruments described in IEC 60651, IEC 60804, IEC 61260 and IEC standards 61672, the GPS(GLOBAL POSITION SYSTEM) function which provides greater consistency in the reports where it is essential to inform the exact location of measurements. The wireless feature can be used widely in airports linking noise to the noisy aircraft for example.

Exclusive registration adjustable from 10 seconds to 1 hour of average values of noise level in the curves frequency weighting A, C and Z and time weighting Fast, Slow and Impulse simultaneously, both maximum values, peak, minimum and relevant statistics (L10 L90 . . . ) with respective date and time.

Time format, date (DD/MM/YYYY) and time (HH:MM:SS) of maximum values in the area monitored by the instrument.

Audio recording from a pre-established rule.

Wireless communication with the controller in order to monitor real time information.

4) Audio Dosimeter: Its use is according ANSI S1.25 Among the innovative features, there is a function that to data noisedosimeter is able to calculate the LEQ Equivalent Level allowing the calculation of worker's daily noise dose regardless of working hours with frequency analysis in bands and third octave, and that allows the doctor or engineer to make the correct choice of earplugs(hearing protectors) or plug insertion through the results in decibels (dB) per frequency band and fractions in order to avoid hearing loss.

5) Vibration meter is used to meet ISO 5349 and ISO 2631

6) Gas monitors—It can be divided into three distinct groups: fixed type, portable and transportable. In plants, fixed type detectors are instruments strategically located near places with imminent risk of gas leaks and/or toxic vapors and/or flammable in cold chambers for monitoring NH3 (ammonia), etc., they prevent workers from contaminated environments to step inside through an audiovisual alarm. Portable detectors meet the requirements of Confined Space, which necessarily requires the use of a gas measurement instrument with direct measuring. Transportable detectors are sometimes classified as CPE—Collective Protection Equipment widely used in plants' maintenance stops.

7) Ionizing radiation alpha, beta, gamma and/or Neutrons detectors—To basically meet the requirements of International Commission on Radiation Protection—ICRP and National Council on Radiation Protection and Measurements NCRP These measurement instruments are divided into fixed types and portable types.

8) Non-ionizing radiation detectors—Primarily, to be used to monitor the microwave radiation generated by antennas of mobile telephone companies easily found in large urban centers,

9) Lux meters—Used to measure the level of lighting of a specific area.

10) Systems IAQ (Indoor air quality)—They are responsible for the systems of measurement parameters.

11) Water quality systems—System used to measure the water PH as well as oxygen level.

Therefore, this is an UNIVERSAL SYSTEM FOR MEASUREMENT INSTRUMENTS FOR INDUSTRIAL HYGIENE AND ENVIRONMENT of great importance for the objective pursued, fully meeting the proposed goals and fulfilling in a practical and efficient manner intended functions, providing advantages inherent to its applicability, innovative and endowed with fundamental requirements of novelty and inventive activity required to deserve patent protection features.

What we claim is:

1. An universal system for measurement instruments for an industrial hygiene and an environment comprising:
a data collector (1) including a portable device or computer equipped with Microsoft Windows or Android Operating System used to communicate with following instruments:
a wet bulb globe temperature thermometer Index (2);
a sound level meter (3);
a noise dosimeter (4);
a vibration meter for hand, arm and whole body (5);
fixed type and portable gas meter detectors (6);
alpha, beta, gamma and/or neutrons ionizing radiation meter (7);
a non-ionizing radiation meter (8);
lux meters (9);
an internal air quality system (10); and
an internal water quality system (11).

2. The universal system according to claim 1, wherein the communication between the data collector and the measurement instruments are performed through wireless technology.

3. The universal system according to claim 1, wherein the universal system monitors simultaneously and in real time, information of heat, noise, gas concentration, vibration, non-ionizing radiation, luminousness, in which workers are exposed.
